(19) United States
(12) Patent Application Publication
(10) Pub. No.: US 2012/0156152 A1
(43) Pub. Date: Jun. 21, 2012

(54) SOFT SOLID ANTIPERSPIRANT COMPOSITIONS

(75) Inventor: James Michael BIANCHI, Trumbull, CT (US)

(73) Assignee: CONOPCO INC. D/B/A UNILEVER, Englewood Cliffs, NJ (US)

(21) Appl. No.: 13/330,838

(22) Filed: Dec. 20, 2011

Related U.S. Application Data
(60) Provisional application No. 61/425,065, filed on Dec. 20, 2010.

(51) Int. Cl. A61K 8/92; A61Q 15/00

(52) U.S. Cl. 424/65

(57) ABSTRACT

A soft solid antiperspirant composition having reduced white marks, the composition comprising:
a) up to 12% by weight of wax structurant, at least a portion of which comprises synthetic ester wax selected from di- and triesters of C₁₂-C₄₀ fatty acids with glycerol or ethylene glycol;
b) carrier oil comprising selected amounts of non-volatile and volatile oil, at least a portion of the non-volatile oil comprising non-volatile ester oil, and
c) at least 18% by weight of astringent antiperspirant active,
   wherein the ratio, by weight, of synthetic ester wax to non-volatile ester oil is from 1:2 to 1:6.5.

This application claims the benefit of U.S. provisional application No. 61/425,065 filed Dec. 20, 2010.

### BACKGROUND OF THE INVENTION

### Field of the Invention

This invention relates to substantially anhydrous soft solid antiperspirant compositions that provide both reduced whitening and desirable sensory properties.

Soft solids are widely used forms of antiperspirant products. The majority of commercially available soft solid products are anhydrous or substantially anhydrous suspensions that comprise antiperspirant active, carrier oil, and structurant. In such products, the antiperspirant active commonly comprises astringent aluminum salt, typically astringent aluminum/zirconium salt, suspended in a matrix formed by a combination of carrier oil and structurant.

Dry, astringent aluminum salts are typically white, powdery materials. The white appearance of the salt contributes to the white deposits on skin and clothes often noted by soft solid users. The tendency towards white deposits (also referred to as “white marks”) increases as the level of antiperspirant active is increased. To a lesser extent white marks can also result from the structurant component of the composition, which often includes white or off-white waxy materials.

Carrier oil can provide a significant benefit in terms of reducing the appearance of white deposits associated with the antiperspirant active. The benefit can, however, be short lived. As the applied composition dries, component materials, especially volatiles, are lost and the composition takes on a white appearance. Drying can also significantly affect a user's perception of a composition's sensory properties. Thus, a user's opinion of a composition that has remained on the skin for some period of time may be very different from the user's opinion of a freshly applied composition.

The challenge to formulators is to extend the time over which a soft solid composition reduces the appearance of white marks while also maintaining desirable sensory properties. In this regard, formulators must additionally contend with the stability issues associated with this particular product form. Compared to solid sticks, by weight, the amount of wax structurant is typically significantly lower in soft solid compositions, and the relative amount of carrier oil to structurant significantly higher. Given these compositional differences, the tendency toward separation of the oil component is generally higher for soft solids than solid sticks. Oil separation can give rise to product instability, and pack leakage. Additionally, stability can be directly impacted by both the choice and level of structurant and carrier oils.

The carrier oil of a soft solid composition is typically a blend of volatile and non-volatile oils. While the non-volatile oil is frequently selected for its masking ability, to impart desirable sensory properties a large portion of the carrier oil is typically comprised of volatile oil. Volatile oil tends to impart a clean, dry feel to the applied composition, as well as to contribute to smooth product application and glide. Additionally, volatile oil aids in fragrance delivery.

In commercially available soft solid compositions, the amount of volatile tends to be relatively high, not only as a percentage of the carrier oil, but also as a function of the total composition. In many commercial products the volatile oil constitutes upwards of 50% by weight of the composition.

Within the industry, the volatile oil of choice is commonly volatile silicone oil, e.g., cyclomethicone, with cyclotetra-, cyclopenta- and cyclohexasiloxanes being among the forms most commonly employed. Cyclomethicone is nominally designated as D4, D5 or D6, depending upon the particular cyclomethicone (e.g., cyclotetra-, cyclopenta-cyclohexasiloxane) predominant therein. The widespread use of cyclomethicone in soft solid antiperspirant suspensions stems, in part, from its solubility and/or compatibility with numerous carrier oils and structurant ingredients, as well as on the ability of the material to contribute a clean, dry, silky feel to the compositions in which it is employed. Compared to many other volatile oils, a significant amount of the cyclomethicone tends to be retained in the packaged suspension composition, as opposed to being lost to evaporation. Volatile retention plays an important role in a product delivering equivalent sensory performance over its useful pack life and is also a factor in product stability, Additionally, the surface tension and spreadability of cyclomethicone contributes to products having a smooth or silky feel on application.

While formulators seeking to maintain reduced whitening over time might consider increasing the amount of non-volatile oil in a composition, this can be problematic in that such an increase must come at the expense of other compositions components. Replacing a portion of the structurant with non-volatile oil is difficult, given the relatively low levels of structurant typical of soft solids, and the stability issues associated with disrupting the structurant/carrier oil balance. Volatile oil is generally present in significant amounts, however, replacing some portion thereof with non-volatile oil can negatively impact both the physical and sensory properties of a soft solid composition. In particular, non-volatile oils can contribute to a composition being perceived by users as greasy, oily, and/or heavy. Moreover, the sensory negatives associated with such components tend to become more apparent as volatile components are lost to evaporation.

Reducing the content of volatile silicone can also have implications in compositions that include silicone elastomer. Silicone elastomer can provide a sensory and/or thickening benefit to soft solids. The elastomer expands in volatile silicones such as cyclomethicone and it is this swelling of the elastomer and the characteristics of the resulting gel that gives rise to its efficacy as a thickening agent and/or sensory enhancer. The swollen elastomer can also be useful in reducing syneresis. Many of the commercially available silicone elastomers are provided in a volatile silicone carrier or are swelled in a suitable medium prior to their use. Replacing a significant portion of volatile silicone oil with non-volatile oil may negatively impact the performance of soft solid compositions that include silicone elastomer.

There remains a need for a stable anhydrous or substantially anhydrous antiperspirant composition that provides desirable sensory properties and reduced white marks for an extended period of time after application.

One aspect of this invention is to provide an anhydrous or substantially anhydrous soft solid antiperspirant composition that overcomes or ameliorates one or more of the issues disclosed above. Another aspect of this invention is to provide such benefits from a composition that optionally includes silicone elastomer.

### SUMMARY OF THE INVENTION

It has now been found that by selecting particular combinations of structurant and oil components and incorporating the components in certain relative amounts, that soft solid compositions meeting one or more aspects of the subject invention are achieved. In one embodiment there is provided an antiperspirant composition comprising:
a) wax structurant, at least a portion of which comprises one or more synthetic ester waxes independently selected from the group consisting of di- and triesters of C₁₂-C₄₀ fatty acids with glycerol or ethylene glycol;
b) carrier oil comprising:
   i. from 50 to 80% by weight, based on the total weight of the carrier oil, of non-volatile oil, at least a portion of which comprises non-volatile ester oil, and
   ii. from 20 to 50% by weight, based on the total weight of the carrier oil, of volatile oil; and
c) at least 18% by weight, based on the total weight of the composition, of astringent antiperspirant active;
   wherein:
   the composition is in the form of a substantially anhydrous soft solid;
   the ratio, by weight, of the synthetic ester wax to the non-volatile ester oil is from 1:2 to 1:6.5; and wax structurant is present in an amount up to 12% by weight, based on the total weight of the composition.

In another embodiment there is provided a method of reducing or controlling perspiration which comprises applying the antiperspirant composition of the subject invention to the underarm area at a dose of from 0.1 to 0.6 grams per underarm.

### DETAILED DESCRIPTION OF THE INVENTION

Soft solids are structured compositions that are generally characterized as having a hardness of from 0.003 to 0.5 Newton/mm², and commonly from 0.003 or 0.01 up to 0.1 Newton/mm². Hardness can be measured using a texture analyzer apparatus which can move a blunt probe into or out from a sample at a controlled speed and at the same time measure the applied force. The parameter which is determined as harness is a function of the force and the projected area of indentation. A specific protocol involves the use of a Stable Micro systems TA.XT2i Texture Analyser. A metal sphere, of diameter 9.5 mm, is attached to the underside of a 5 kg load cell, and positioned just above the sample surface. Under control of Expert Exceed™ software, the sphere is indented into the sample at an indentation speed of 0.05 mm/s for a distance of 7 mm and reversed to withdraw the sphere from the sample at the same speed. Data comprising time(s) distance (mm) and force (N) is acquired at a rate of 25 Hz. The hardness H at a penetration of 4.76 mm is calculated using the formula:

H=F/A

in which H is expressed in N·mm⁻², F is the load at the same travelled distance in N, and A is the projected area of the indentation in mm². This area can be calculated geometrically and is equal to the area of a diametral plane of the sphere, i.e., π×(4.76)² mm².

The term “anhydrous” as applied to the subject compositions means that no separate aqueous liquid phase is present and that the antiperspirant composition is free of water, exclusive any bound or complexed water that may be present in the raw materials, such as, for example, any water of hydration in the antiperspirant active. The term “substantially anhydrous” means, based on the total weight thereof, the antiperspirant composition contains less than 2% by weight of added water, exclusive of any bound or complexed water that may be present in the raw materials. In a preferred embodiment, the antiperspirant composition contains less than 1% of added water. In one embodiment of interest, the antiperspirant composition contains less than 0.5% by weight of added water. Bound or complexed water present in the raw materials is not considered to be “added water” as such term is used herein. Unlike emulsions and other multiple phase compositions with separate internal and external phases, the subject compositions are desirably single phase compositions.

### Antiperspirant Active

The composition desirably contains a relatively high content of antiperspirant active. Antiperspirant active is preferably incorporated in an amount of at least 18% by weight, more particularly from 20 to 30% of the weight of the composition, based on the total weight thereof. In at least one embodiment of interest the antiperspirant active is present in an amount of from 22 to 27% by weight of the composition.

Antiperspirant actives for use herein are often selected from astringent active salts, including, in particular, aluminum, zirconium and mixed aluminum/zirconium salts, including both inorganic salts, salts with organic anions and complexes. Preferred astringent salts include aluminum, zirconium and aluminum/zirconium halides and halohydrate salts, such as chlorohydrates and activated aluminum chlorohydrates.

Aluminum halohydrates are usually defined by the general formula Al₂(OH)ₓQ_{y}.wH₂O in which Q represents chlorine, bromine or iodine, x is variable from 2 to 5 and x+y=6 while wH₂O represents a variable amount of hydration.

Zirconium actives can usually be represented by the empirical general formula: ZrO(OH)_{2n-nz}B_{z}.wH₂0 in which z is a variable in the range of from 0.9 to 2.0 so that the value 2n-nz is zero or positive, n is the valency of B, and B is selected from the group consisting of chloride, other halide, sulfamate, sulfate and mixtures thereof. Possible hydration to a variable extent is represented by wH₂O. In one embodiment B represents chloride and the variable z lies in the range from 1.5 to 1.87. In practice, such zirconium salts are usually not employed by themselves, but as a component of a combined aluminum and zirconium-based antiperspirant.

The above aluminum and zirconium salts may have coordinated and/or bound water in various quantities and/or may be present as polymeric species, mixtures or complexes. In particular, zirconium hydroxy salts often represent a range of salts having various amounts of the hydroxy group. Aluminum zirconium chlorohydrate may be particularly preferred.

Antiperspirant complexes based on the above-mentioned astringent aluminum and/or zirconium salts can be employed. The complex often employs a compound with a carboxylate group, and advantageously this is an amino acid. Examples of suitable amino acids include dl-tryptophan, dl-β-phenylalanine, dl-valine, dl-methionine and β-alanine, and preferably glycine which has the formula CH₂(NH₂)COOH.

It is highly desirable to employ complexes of a combination of aluminum halohydrates and zirconium chlorohydrates together with amino acids such as glycine, examples of which are disclosed in U.S. Pat. No. 3,792,068 (Luedders at al). Certain of those Al/Zr complexes are commonly called AZG in the literature. AZG actives generally contain aluminum, zirconium and chloride with an Al/Zr ratio in a range from 2 to 10, especially 2 to 6, an Al/Cl ratio from 2.1 to 0.9 and a variable amount of glycine. Actives of this type are available from suppliers that include Summit Reheis. In one preferred embodiment the active is enhanced activity or activated aluminum/zirconium halohydrate, in particular, activated aluminum-zirconium tetrachlorohydrex glycine (AAZG).

Other actives which may be utilized include astringent titanium salts, for example those described in GB 2299506A.

The proportion of solid particulate antiperspirant salt in a suspension composition normally includes the weight of any water of hydration and any complexing agent that may also be present in the solid active.

In one or more embodiments it is desirable that the mean particle size of the antiperspirant salts is within the range of 0.1 to 100 micron with a mean particle size that is often from 3 to 30 microns, more particularly from 5 to 35 microns, and certain embodiments of interest from 10 to 25 microns. Actives having either larger or smaller mean particle sizes can also be contemplated.

The particulate antiperspirant active may be present in the form of hollow spheres or dense particles (by which is meant particles which are not hollow) at the discretion of the manufacturer. To reduce the appearance of visible deposits on the skin to which the composition is applied or on clothing which comes into contact with the composition, it is preferable for the particles to be substantially free from hollows. Hollows can be eliminated by crushing the spheres.

The composition takes the form of a suspension in which antiperspirant active in particulate form is suspended in the matrix formed by the combination of structurant and carrier oil.

### Wax Structurant

This term “wax” is applied herein to a variety of materials including mixtures which have similar physical properties, namely that they are solid materials that are firm to brittle hard, malleable at 20° C. and melt to a mobile liquid at a temperature above 40° C. but generally below 95° C.; additionally, such materials are water-insoluble and remain water-immiscible when heated above their melting points.

From the perspective of processibility and crystallization behavior it is often desirable that the waxes employed herein have melting points within the range of 55° C. to 80° C., inclusive.

Waxes are herein classified as “natural” or “synthetic” waxes. As applied to waxes, the term “natural” refers to waxes that are animal, vegetable or mineral in origin, including waxes that are refined or otherwise treated to remove contaminants or purify. The term “synthetic” as applied to waxes refers to waxes that are synthesized from non-wax starting materials or that are produced by the chemical modification of a wax starting material, the later commonly being a subclass of synthetic waxes known as “semi-synthetic waxes”.

The wax structurant of the subject compositions comprises one or more synthetic ester waxes. Desirably the waxes are one or more waxes independently selected from the group consisting of di- and triesters of long chain fatty acids with glycerol or ethylene glycol. As used herein, unless otherwise specified, the term “long chain” as applied to fatty materials refers to chains of 12 or more carbon atoms, with materials having carbon chains of C₁₂-C₄₀ being common for such materials. The feedstock from which such esters are derived are generally mixtures comprising long chain fatty materials having chain lengths and chain length distributions that depend, in large part, on the source of the fatty material and the treatment thereof. A synthetic ester wax of particular interest is derived from montan wax. Raw montan wax is a fossil vegetable wax that is high in impurities and contaminants. Through a series of processing steps, the raw montan wax is converted to a fatty acid feedstock that is reacted with a polyvalent alcohol such as ethylene glycol or propylene glycol to produce di- and triesters of interest herein.

It is also possible to employ a synthetic fatty acid feedstock in the production of the subject ester waxes. Irrespective of the source thereof, it is generally desirable that the fatty groups of the feedstock material are comprised primarily of linear, aliphatic carbon chains. The carbon chains of such fatty groups are primarily even-numbered and preferably are saturated. In at least one embodiment it is desirable that a plot of the chain length distribution of the fatty material as a function of its percent by weight of the composition has a maximum at C₂₆-C₃₀ or C₂₈-C₃₀.

Chain length distributions in which the difference between the longest and shortest carbon chains (hereinafter also referred to as the “chain length spread”) is 8 or more carbons and, more particularly, 10 or more carbons, are not uncommon, with differences of from 10 to 30 carbons, more particularly from 14 to 26 carbon atoms being of particular interest in at least one embodiment. Without wishing to be bound by theory, such distributions may provide for more stable compositions. For example, a typical chain length distribution of raw, naturally occurring montan wax is C₂₂ to C₃₄, for both the fatty alcohol and fatty ester components thereof, which distribution is subject to variation given the source thereof. In producing a feedstock material from naturally occurring, raw montan wax, carbon chains can be split as well as dimerized; thus, the material that is ultimately esterified with the polyvalent alcohols may have a broader or narrower distribution, depending upon the particular raw material source and processing conditions employed. C₁₆₋₃₆ and C₁₈₋₃₈ feedstocks are of interest in one or more embodiments.

Of particular interest are synthetic waxes selected from the group consisting of ethylene glycol diesters of saturated C₁₈₋₃₆ fatty acid waxes, triglycerides of C₁₈₋₃₆ saturated fatty acid waxes, and mixtures thereof.

Synthetic ester waxes are commercially available from numerous suppliers including Croda, Koster Kuenen, and Clariant. A number of suitable materials are available from Croda under the trademark “Syncrowax” with the waxes known as Syncrowax ERLC (identified as the ethylene glycol di-ester of C18-36 fatty acids) and, Syncrowax HGLC (identified as the triglyceride ester of C18-36 fatty acids) being of particular interest.

In one or more embodiments the synthetic ester wax comprises at least 30% by weight and, more particularly, at least 40° by weight of the wax structurant. In other embodiments the synthetic ester wax comprises at least 50% by weight of the wax structurant.

Wax structurant is desirably employed in the subject compositions in amounts up to 12% by weight. The preferred amount of wax structurant of depends, in part, on the particular wax structurant employed and the amount, if any, of silicone elastomer and/or other non-wax structuring materials present. In many compositions wax structurant is present in an amount of from 3 to 10% by weight, more particularly from 5 to 8% by weight.

In addition to synthetic ester wax, the subject compositions may include other co-structurant waxes. Preferably the additional wax is an organic wax. Hydrocarbon waxes, such as, for example paraffin wax, microcrystalline wax and polyethylene waxes are of particular interest as one or more additional wax components. The polyethylene waxes of interest typically have weight average molecular weights of from 200 to 2000, more particularly, from 200 to 1000, and even more particularly from 300 to 600. The co-structurant wax may be natural or synthetic. While natural ester waxes such as, for example castor wax, may be present, it is preferable that the total amount thereof does not exceed 4% by weight of the total weight of the composition and preferably does not exceed 2% by weight or, more particularly, does not exceed 1% by weight of the composition. Linear fatty alcohol, especially fully saturated alcohol containing 14 to 24 carbon atoms, such as, for example cetyl alcohol, stearyl alcohol, cetearyl alcohol, behenyl alcohol, and the like, may be employed as a co-structurant, however, it is preferred that the total amount thereof does not exceed 2% by weight or, more particularly, does not exceed 1% by weight, based on the total weight of the composition.

### Carrier Oil

The water-immiscible carrier oil herein comprises a mixture of materials, which are relatively hydrophobic so as to be immiscible in water, which materials are liquid at 20° C. up to at least the temperature at which the structurant is dissolved or dispersed therein. Melting point data as well as information as to whether a material is or is not water-immiscible is available from numerous literature sources, for example, the CRC Handbook of Chemistry and Physics published by CRC Press. For any material where such data is not available in the literature, it can be measured simply by any chemist using conventional techniques.

As used herein the term “volatile” is used to designate a material having a measurable vapor pressure at 25° C. Typically the vapor pressure of a volatile oil lies in a range of at least 1 Pa or preferably at least 10 Pa at 25° C., though generally will be less than 4 kPa (30 mmHG). A non-volatile oil can be considered to generate a vapor pressure of below 1 Pa at 25° C.

The volatile oil component of the subject invention comprises from 20% to 50% by weight, more particularly from 25 to 45% by weight, even more particularly, from 35 to 45% by weight of the carrier oil. Especially desirably as volatile oil is volatile silicone oil. Volatile silicone oils suitable for use herein can be linear or cyclic polyorganosiloxanes or mixtures thereof. Preferred cyclic siloxanes include polydimethylsiloxanes and particularly those containing from 3 to 9 silicone atoms and preferably not more than 7 silicone atoms and most preferably from 4 to 6 silicon atoms, otherwise often referred to as cyclomethicones. Preferred linear siloxanes include polydimethylsiloxanes containing from 3 to 9 silicon atoms. The volatile siloxanes normally by themselves exhibit viscosities of below 10⁻⁵ m²/sec (10 centistokes), and particularly above 10⁻⁷ m²/sec (0.1 centistokes), the linear siloxanes normally exhibiting a viscosity of below 5×10⁻⁶ m²/sec (5 centistokes). The volatile silicone oils can also comprise branched linear or cyclic siloxanes such as the aforementioned linear or cyclic siloxanes substituted by one or more pendant —O—Si(CH₃)₃ groups. Examples of commercially available volatile silicone oils having grade designations 344, 345, 244, 245 and 246 from Dow Corning Corporation. In at least one embodiment of particular interest, the volatile silicone oil comprises cyclomethicone and preferably comprises cyclopentasiloxane and/or cyclopentasiloxane.

In addition to the volatile silicone oil, non-limiting examples of other volatile oils that may be present in the subject compositions are volatile hydrocarbons such as, for example, volatile water-immiscible materials comprising a hydrocarbon chain which optionally can further comprise and embedded ether or ester linkage. It is especially desirable that at least 50% by weight, more particularly, at least 60% by weight, even more particularly, at least 70% by weight of the volatile oil is volatile silicone oil.

The non-volatile oil component of the subject invention comprises from 50 to 80% by weight, more particularly from 55 to 75% by weight, even more particularly, from 55 to 65% by weight of the carrier oil. The non-volatile oils are preferably liquid at 15° C., with oils having a boiling point of at least 150° C. being particularly advantageous.

At least a portion of the non-volatile oil comprises ester oil. Ester oils represent a particularly useful class of non-volatile oil. The ester oils can be aliphatic or aromatic. Many desirable aliphatic esters contain at least one hydrocarbon chain of 8 or more carbons, for example chains of from 8 to 25 carbons, derived from a monohydric alcohol or monocarboxylic acid. Suitable aliphatic esters can be derived from monohydric alcohols such as selected from C₁ to C₂₀ alkanols esterified with a carboxylic acid selected from C₈ to C₁₀ alkanedioic acids. Such esters include isopropyl myristate, lauryl myristate, isopropyl pamitate, diisopropyl sebacate and diisopropyl adipate. Other suitable ester oils include glyceride oils and in particular triglyceride oils derived from glycerol and fatty acids, sometimes olefinically unsaturated rather than saturated, containing at least 6 carbons and especially natural oils derived from unsaturated carboxylic acids containing from 16 to 20 and especially 18 carbons.

Aromatic ester oils include as a subset thereof esters with both aromatic and aliphatic groups, i.e., aliphatic/aromatic ester oils. Among the suitable aromatic ester oils are oils derived from benzoic acid. Examples include C₈ to C₁₈ alkyl benzoates or mixtures thereof, including in particular C₁₂ to C₁₅ alkyl benzoates. Many suitable benzoate esters are available under the trademark Finsolv from Innospec Performance Chemicals. It is desirable to consider the non-volatile ester oil in relation to the amount of the non-volatile synthetic ester wax present in the composition. Desirably, the ratio, by weight of the synthetic ester wax to non-volatile ester oil is from 1:2 to 1:6.5, with ratios of from 1:2 to 1:6 and, more particularly, from 1:2.5 to 1:5.5 being of interest in one or more embodiments.

The non-volatile oil may optionally include one or more oils in addition to non-volatile ester oil. Non-limiting examples of other classes of materials from which suitable masking oils can be found include ether oils and hydrocarbon oils.

Ether oils represent further examples of suitable non-volatile oils. Preferably, the ether oils contemplated for use herein comprise liquid aliphatic ethers derived from a polyglycol especially from polypropylene glycol (PPG), the latter preferably containing at least 3 mers, such as 3 to 20, with a monohydric alcohol. The monohydric alcohol often contains between 3 and 20 carbons. As the molecular weight of the PPG increases, so the chain length of the monohydric alcohol can decrease. For example, suitable ether oils can vary between a low molecular weight PPG with a long chain fatty alcohol, such as PPG-3 myristyl ether and lower alkyl ethers of a higher molecular weight PPG, such as the ether named PPG-14 butyl ether in the CFTA Handbook.

Suitable hydrocarbon oils are commonly selected from mineral oils, hydrogenated polydecene and hydrogenated polyisobutene. Hydrocarbon oils are desirable in that they, like most of the other non-volatile oils described herein, also function as emollients and have a soothing, softening effect on skin.

Optionally, the carrier oil may further comprise one or more non-volatile silicone oils. Illustrative, non-limiting examples of non-volatile silicone oils suitable for use in the practice of this invention are: polyalkyl siloxanes, polyalkylaryl siloxanes and polyethersiloxane copolymers. These can suitably be selected from dimethicone and dimethicone copolyols. Commercially available non-volatile silicone oils include products available from suppliers that include Dow Corning

In at least one preferred embodiment the non-volatile oil comprises a mixture of ester oil and ether oil. In one embodiment of interest the mixture of ester oil and ether oil comprises at least 80% by weight, preferably at least 90% by weight of the non-volatile oil present in the composition.

### Silicone Elastomers

The silicone elastomers impart a silky feel to the compositions of the invention and also contribute to reducing syneresis of the final product. Silicone elastomers for use herein include cross-linked polydimethyl or polymonomethyl siloxanes optionally having end groups such as hydroxyl or methyl. Such elastomers are commercially available from numerous sources and can be readily made using conventional techniques well known to those skilled in the art.

Preferred silicone elastomers for use in the invention are polydiorganosiloxanes, preferably derived from suitable combinations of R₃SiO_{0.5} units and R₂SiO units where each R independently represents an alkyl, alkenyl (e.g. vinyl), alkaryl, aralkyl, or aryl (e.g. phenyl) group. R is most preferably methyl.

The preferred cross-linked silicone elastomers of the Invention are cross-linked polydimethyl siloxanes (which have the CTFA designation dimethicone), optionally having end groups such as hydroxyl or methyl.

One preferred elastomer of the invention is DC 9040, an example of a non-emulsifying elastomer. DC 9040 cross-linking chemistry is as follows:

The cross linker used in DC 9040 is an alpha, omega aliphatic diene of the following structure:

CH₂═CH(CH₂)ₓCH═CH₂

where x ranges from 1-20. A gel is formed by crosslinking and addition of Si—H across double bonds in the alpha, omega-diene. The following Dow Corning patent describes the DC 9040 elastomer: U.S. Pat. No. 5,654,362.

Another preferred elastomer which can be used in compositions of the invention is SFE 839 from General Electric.

Yet another elastomer which can be used in compositions of the invention is DC 3-2365. The structure of the cross-linker used in DC3-2365 is given below:

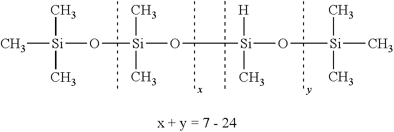

Another preferred elastomer of the invention is Silicone/Urethane Copolymer. The structure of the urethane cross-linker is given below:

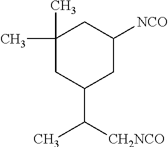

The tradename for the silicone-urethane copolymer is Polyderm PP I-SI-100. The supplier is Alzo Incorporated, Matawan, N.J.

Other preferred elastomers are the following: an elastomeric resinous material which is a silicone polymer having a) a backbone with the following structure: R₃SiO(R′₂SiO)ₘ(R″R′″SiO)ₙSiR₃, where m is 1-250, n is 0-250, R, R″, R″ are alkyl groups containing 1-6 carbon atoms, and R′″ is CH₂═CHCH₂O(CH₂CHₓO)ₓ(CH(CH₃)CH₂O)_{y}H and x+y is less than or equal to thirty; and b) the polymer backbone is crosslinked with one or more of the following compounds: an alpha-omega diene whose structure is CH₂═CH(CH₂)_{z}CH═CH₂; an alpha-omega diyne whose structure is CH≡C(CH₂)_{z}C≡CH, and an alpha-omega ene-yne whose structure is CH₂═CH(CH₂)_{z}C≡CH, where z ranges from one to twenty.

Other preferred elastomers are the following: an elastomer as described just above selected from the group consisting of: a silicone gel having a cross-linked polymer structure with 20 mol % substitution of the group defined by R′″, wherein x=6 and y=0; and a silicone gel having a cross-linked polymer structure with 20 mol % substitution of the group defined by R′″, wherein x=11 and y=0.

Other preferred elastomers are the following: Dow Corning cross-linked, ethoxylated silicone gels branded as DC 9010 or a combination of such gels.

The degree of cross-linking of the silicone elastomers is suitably from about 0.05% to about 35%, preferably being in the range of about 0.15% to about 7%, more preferably from about 0.2 to about 2%.

When present, elastomer is typically included in amounts of from 0.001 to 2% by weight or greater, more particularly, from 0.01 to 1% by weight, based on the total weight of the composition. In one or more embodiments, compositions that contain 0.5% by weight or more of silicone elastomer are of particular interest.

### Optional Ingredients

The compositions of this invention may include one or more non-wax rheology modifiers which add thixotropic body or aid in controlling syneresis. Such materials may also assist in processing of the composition while it is in molten form before being filled into molds. Non-limiting examples of such rheology modifiers include, for example, aluminum stearate, stearamide MEA, silica, in particular, finely divided silica such as fumed or precipitated silica, talc, and mixtures thereof. Silica is among the preferred rheological additives in one or more embodiments.

When present, rheology modifiers are desirably included in compositions of the invention in amounts, based on the total weight of the composition, of up to 4.0% by weight, with amounts of from of from 0.05 to 2.0% by weight, more particularly from 0.1 to 1.5% by weight being of interest in one or more embodiments.

Other ingredients, conventional in the art of soft solid antiperspirant compositions may be included in the compositions of the present invention. Optional ingredients include wash-off agents, often present in the subject compositions an amount of at least 0.05% by weight, and advantageously at least 0.25% by weight up to 5% by weight to assist in the removal of the composition from skin or clothing. When present, the wash-off agent is often present in an amount up to 1%. Such wash-off agents are typically nonionic surfactants such as esters or ethers containing both a C₈ to C₂₂ alkyl moiety and a hydrophilic moiety which can comprise a polyoxyalkylene group (POE or POP) and/or a polyol, e.g., glycerol or sorbitol.

Fragrance is another common optional component. For purposes of this invention, unless otherwise indicated, fragrance is considered as a separate component from the carrier oil, and the amount thereof is not included as the part of the amount of carrier oil permitted in the subject compositions. The total amount of fragrance (inclusive of all material present as part of fragrance encapsulate) is often from 0.001 to 5 wt. %, based on the total weight of the composition In one embodiment, fragrance is desirably employed at a level of from 0.05 to 4 wt. %, more particularly from 0.1 to 3.5 wt %, based on the total weight of the composition. Encapsulated fragrance may be formulated as shear or moisture sensitive materials.

Non-limiting examples of other optional ingredients are drying agents, such as talc or aluminum starch octenylsuccinic, skin benefit agents such as allantoin, vitamins or lipids: colors; preservatives; skin cooling agents such as menthol and menthol derivatives; skin feel improvers such as finely divided high melting point polyethylene, micro-fine aluminum oxide powder and/or a particulate polymethylmethacrylate such as Ganzpearl® GMX-0810 from Ganz Chemcal.

The amount of such optional adjuncts should not negatively impact the total solid content desired in the subject compositions. When present, the total amount of such optional ingredients typically does not exceed 10% by weight of the composition and often does not exceed 5% by weight of the composition.

If desired the composition can comprise a supplementary deodorant active, i.e., an active other than the antiperspirant salt. Suitable supplementary deodorant actives can comprise deodorant effective concentrations of deoperfumes, and/or microbicides, including particularly bactericides, such as chlorinated aromatics, including biguanide derivatives, of which materials known as triclosan (Irgasan® DP300 from Ciba Specialty Chemicals), tricloban and chlorhexidine warrant specific mention. Supplementary deodorant actives are commonly employed at a concentration of from 0.1 to 5% by weight and often up to 1% by weight of the composition.

Of particular interest in one or more embodiments is an antiperspirant composition comprising:
a) wax structurant, at least a portion of which comprises one or more synthetic ester waxes independently selected from the group consisting of di- and triesters of C₁₂-C₄₀ fatty acids with glycerol or ethylene glycol;
b) carrier oil comprising:
   i. from 50 to 80% by weight, based on the total weight of the carrier oil, of non-volatile oil, at least a portion of which comprises non-volatile ester oil, and
   ii. from 20 to 50% by weight, based on the total weight of the carrier oil, of volatile oil;
c) at least 18% by weight, based on the total weight of the composition, of astringent antiperspirant active; and
d) silicone elastomer;
   wherein:
   the composition is in the form of a substantially anhydrous soft solid;
   the ratio, by weight, of the synthetic ester wax to the non-volatile ester oil is from 1:2 to 1:6.5; and wax structurant is present in an amount up to 10% by weight, based on the total weight of the composition.

The compositions of the instant invention can be prepared by a conventional process in which the wax structurant is dissolved or dispersed in the carrier fluid at a temperature above the melting point of the wax, the particulate antiperspirant active material is introduced into the mixture of carrier oils and structurant and the resultant composition is cooled to below its normal setting temperature, thereby forming a soft solid which can be caused to flow by the application of gentle pressure. The antiperspirant active is often introduced at a temperature intermediate between that at which the wax structurant is dispersed in the carrier oils and that at which the composition sets, such as at a temperature in the range of about ⅓^{rd} to ⅔^{rd} above the setting temperature, for example, at about 65° C. if the carrier oil/structurant mixture is formed at 80° C. and the composition sets at 50° C.

Desirably the composition is introduced while it is still mobile into the storage chamber of the container from which it is dispensed and thereafter is cooled or allowed to cool. The compositions may be employed in dispensers suitable for use with soft solid compositions.

Except in the operating and comparative examples, or where otherwise explicitly indicated, all numbers in this description indicating amounts or ratios of materials, conditions of reaction; physical properties of materials and/or use; dimensions and dimension ratios, are to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above. It should be noted that in specifying any range of concentration or amount, any particular upper concentration or amount can be associated with any particular lower concentration or amount.

All parts, percentages, ratios, and proportions referred to in the subject specification and in the appended claims are by weight unless otherwise indicated.

The following Examples will more fully illustrate the embodiments of this invention. The examples are not intended to limit the scope of the invention in any manner.

### EXAMPLES

Compositions having the formulations described in Table 1 were prepared and introduced to a soft solid dispenser having a plurality of dispensing openings and suitable for the application of a target dose of 0.4 g of product per underarm. A commercially available soft solid was employed as C3.

TABLE 1

| Ingredient | Example | Example | Example | Example | Example | | |
|---|---|---|---|---|---|---|---|
| (% by weight) | 1 | 2 | 3 | 4 | 5 | C1 | C2 |
| Cyclopentasiloxane | balance | balance | balance | balance | balance | balance | balance |
| | to 100 | to 100 | to 100 | to 100 | to 100 | to 100 | to 100 |
| C18-36 Acid | 3.5 | 3.5 | 3.5 | 3.5 | 3.5 | 3.5 | 3.5 |
| Triglyceride | | | | | | | |
| (Syncrowax ™ HPLC) | | | | | | | |
| Microcrystalline Wax | 3.5 | 3.5 | 3.5 | 3.5 | 3.5 | 3.5 | 3.5 |
| Dimethicone (50 cst) | — | — | — | — | — | — | 8.0 |
| PPG-14 Butyl Ether | 19.0 | 28.5 | 28.5 | 19.0 | 22.0 | 9.5 | — |
| C12-15 Alkyl | 19.0 | 9.5 | 9.5 | 19.0 | 16.0 | 28.5 | — |
| Benzoate | | | | | | | |
| Silicone Elastomer* | 4.0 | 4.0 | 4.0 | 4.0 | 4.0 | 4.0 | 4.0 |
| BHT | 0.05 | 0.05 | 0.05 | 0.05 | 0.05 | 0.05 | 0.05 |
| AAZG | 26.3 | 26.3 | 26.3 | 26.3 | 26.3 | 26.3 | 26.3 |
| Silica | 1.0 | 1.0 | 1.50 | 1.50 | 1.50 | 1.0 | 1.0 |
| Fragrance | 1.3 | 1.3 | 1.3 | 1.3 | 1.3 | 1.3 | 2.5 |

*DC 9040 Silicone Elastomer from Dow Corning, a mixture of silicone elastomer in cyclomethicone.

Within 24 hours of manufacture, the composition identified as C1 exhibited leaking of silicone oil when maintained at room temperature. Examples 1 to 5 and C2 did not.

The compositions were evaluated for whiteness pursuant to the following protocol;

An 11″ (28 cm)×9″ (23 cm) sheet of gray 1200 grit waterproof sandpaper was positioned horizontally such that the width across the top of the paper was 11″ (28 cm). Samples of four different compositions (0.30 g±0.01 g per sample) were spaced across the width of the sandpaper sheet, approximately 1 cm from the top edge of the sheet. The sheet was positioned approximately 200 μm from the lower surface of the stainless steel bar of Accu-Lab Drawdown Machine (bar diameter 0.5″ (1.3 cm)), and the bar was roped from the top to the bottom edge of the sheet, and then back from the bottom to the top edge of the sheet to deposit the samples on the sandpaper in a controlled manner in individual stripes.

Five minutes of the application, a black polyester/cotton cloth was placed on the sandpaper sheet over the sample stripes being evaluated. A rub-off application was performed by pulling the cloth over the stripes with controlled weight (160 gm); and speed (5 seconds).

L* value measurements were taken of the residue rubbed off the sandpaper and onto the black cloth using a hand-held Minolta Chromameter set to read in the L*a*b* measuring mode. A higher L* value indicates more whiteness than a lower L* value. Measurements were taken at four discrete points per sample residue, and three replicate applications were performed for each of the compositions tested. The L.* values are a reported as the mean value of 12 measurements per composition rested.

Two sets of measurements were taken (i.e.; immediately after application of the residue to the cloth, which measurement is nominally referred to as whiteness after 5 minutes, and 2 hours after application of the residue to the cloth). The values obtained are reported in Tables 2 and 3. A Tukey HSD statistical analysis of the L* values was carried out at the 95% confidence level. Statistical significance is reported together with the mean L* values. Entries sharing a letter are not statistically different at the 95% confidence level.

TABLE 2
WHITENESS AFTER 5 MINUTES - RUB-OFF ON CLOTH

| | Mean Rub-Off Value | Statistical Significance | | | |
|---|---|---|---|---|---|
| Composition | (L* value) | (95% confidence) | | | |
| C2 | 20.60 | A | | | |
| C3 | 17.86 | | B | | |
| Example 3 | 17.39 | | B | C | |
| Example 4 | 17.12 | | B | C | |
| Example 2 | 16.80 | | B | C | D |
| Example 1 | 15.97 | | | C | D |
| C1 | 15.57 | | | | D |

TABLE 3
WHITENESS AFTER 2 HOURS - RUB-OFF ON CLOTH

| | Mean Rub-Off | Statistical Significance | | |
|---|---|---|---|---|
| Composition | value | (95% confidence) | | |
| C2 | 27.68 | A | | |
| C3 | 21.12 | | B | |
| Example 3 | 17.14 | | | C |
| Example 4 | 16.76 | | | C |
| Example 2 | 16.54 | | | C |
| Example 1 | 15.94 | | | C |
| C1 | 15.69 | | | C |

After two hours, Example 1 to 4 and C1 all showed improved whitening over the C2 and C3 compositions. Further the difference between the 2 hour and 5 minute rub-off values was significantly less for Examples 1 to 4 and C1 than for the C2 and C3 compositions.

Sensory profiling of the Example 3, Example 5, C2 and C3 compositions was carried out in a trained panel test (9 female panelists). In this evaluation, the compositions were applied at a target dose of 0.4 g per armpit. Results of the evaluation are reported in Table 4. A Tukey HSD statistical analysis of the data was carried out at the 95% confidence level. Statistical significance is reported together with the mean rub-off values. Entries sharing a letter are not statistically different at the 95% confidence level.

TABLE 4
Sensory Profiling

| | | | | |
|---|---|---|---|---|
| COMPOSITION | Example 3 | Example 5 | C2 | C3 |

During Application

| | | | | | | | | |
|---|---|---|---|---|---|---|---|---|
| Coolness | 1.9 | | 1.6 | | 1.7 | | 1.7 | |
| Force to Apply | 3.0 | b | 3.0 | b | 2.9 | b | 2.8 | b |
| Slipperiness (Product v | 5.7 | a | 5.7 | a | 5.9 | a | 6.0 | a |
| Skin) | | | | | | | | |
| Fordce to Spread | 3.0 | b | 2.9 | b | 2.8 | b | 2.8 | b |
| Crumbling | 0.3 | | 0.3 | | 0.2 | | 0.2 | |
| Slipperiness (Product v | 5.8 | a | 6.0 | a | 6.0 | a | 6.1 | a |
| Product | | | | | | | | |
| Residue | 2.0 | bc | 2.1 | abc | 2.3 | a | 2.1 | ab |

Immediately after Application

| | | | | | | | | |
|---|---|---|---|---|---|---|---|---|
| Dryness | 7.2 | b | 7.3 | b | 7.1 | b | 7.2 | b |
| Coolness | 1.2 | | 1.0 | | 1.2 | | 1.0 | |
| Whiteness | 0.8 | c | 1.2 | bc | 1.7 | ab | 2.3 | a |
| Shine | 3.4 | a | 3.5 | a | 3.2 | ab | 3.4 | a |
| Visual Texture | 0.3 | c | 0.8 | bc | 1.2 | ab | 1.6 | a |
| Stickiness | 0.5 | | 0.5 | | 0.5 | | 0.4 | |
| Slipperiness | 7.0 | bc | 7.1 | ab | 7.2 | a | 7.2 | a |
| Residue | 1.8 | | 1.7 | | 1.9 | | 2.0 | |

At 2 Minutes

| | | | | | | | | |
|---|---|---|---|---|---|---|---|---|
| Dryness | 7.6 | | 7.6 | | 7.5 | | 7.6 | |
| Coolness | 0.8 | | 0.7 | | 0.9 | | 0.8 | |
| Stickiness | 0.5 | | 0.4 | | 0.4 | | 0.3 | |
| Slipperiness | 6.9 | c | 7.0 | bc | 7.2 | a | 7.1 | ab |
| Residue | 1.7 | bc | 1.5 | c | 1.8 | ab | 1.9 | a |

At 4 Minutes

| | | | | | | | | |
|---|---|---|---|---|---|---|---|---|
| Dryness | 7.9 | | 8.0 | | 7.9 | | 7.9 | |
| Coolness | 0.6 | | 0.5 | | 0.7 | | 0.6 | |
| Stickiness | 0.4 | | 0.3 | | 0.3 | | 0.2 | |
| Slipperiness | 6.9 | b | 6.9 | b | 7.1 | a | 7.2 | a |
| Residue | 1.5 | bc | 1.4 | c | 1.6 | ab | 1.7 | a |

At 6 Minutes

| | | | | | | | | |
|---|---|---|---|---|---|---|---|---|
| Dryness | 8.4 | ab | 8.5 | a | 8.4 | ab | 8.2 | b |
| Coolness | 0.5 | | 0.4 | | 0.5 | | 0.5 | |
| Stickiness | 0.3 | | 0.3 | | 0.2 | | 0.2 | |
| Slipperiness | 6.9 | bc | 6.8 | c | 7.0 | ab | 7.1 | a |
| Residue | 1.5 | | 1.4 | | 1.5 | | 1.6 | |

At 10 Minutes

| | | | | | | | | |
|---|---|---|---|---|---|---|---|---|
| Dryness | 8.7 | | 8.8 | | 8.8 | | 8.7 | |
| Coolness | 0.3 | | 0.2 | | 0.3 | | 0.2 | |
| Whiteness | 0.1 | c | 0.2 | bc | 0.7 | a | 0.7 | a |
| Visual Texture | 0.0 | c | 0.1 | bc | 0.5 | a | 0.5 | a |
| Stickiness | 0.4 | | 0.3 | | 0.2 | | 0.2 | |
| Slipperiness | 6.9 | | 6.9 | | 7.0 | | 7.0 | |
| Residue | 1.4 | | 1.2 | | 1.4 | | 1.5 | |

The Example 3 and Example 5 compositions were both found to be significantly less whitening than the C2 and C3 compositions. In such testing, the Example 3 and Example 5 compositions were also found to have sensory properties that were generally comparable to C2 (which had a significantly higher level of volatile carrier oil) and C3.

1. An antiperspirant composition comprising:
a) wax structurant, at least a portion of which comprises one or more synthetic ester waxes independently selected from the group consisting of di- and triesters of C₁₂-C₄₀ fatty acids with glycerol or ethylene glycol;
b) carrier oil comprising:
i. from 50 to 80% by weight, based on the total weight of the carrier oil, of non-volatile oil, at least a portion of which comprises non-volatile ester oil, and
ii. from 20 to 50% by weight, based on the total weight of the carrier oil, of volatile oil; and
c) at least 18% by weight, based on the total weight of the composition, of astringent antiperspirant active;
wherein:
the composition is in the form of a substantially anhydrous soft solid;
the ratio, by weight, of the synthetic ester wax to the non-volatile ester oil is from 1:2 to 1:6.5; and wax structurant is present in an amount up to 12% by weight, based on the total weight of the composition.

2. A composition as described in claim 1 wherein the synthetic ester wax is one or more ester waxes independently selected from the group consisting of ethylene glycol diesters of saturated C₁₈₋₃₆ fatty acid waxes and triglycerides of C₁₈-C₃₆ saturated fatty acid waxes.

3-18. (canceled)

19. A composition as described in claim 1 wherein the synthetic ester wax comprises at least 40% by weight of the wax structurant.

20. A composition as described in claim 1 that further comprises one or more hydrocarbon waxes.

21. A composition as described in claim 1 that further comprises microcrystalline wax.

22. A composition as described in claim 1 that further comprises silicone elastomer.

23. A composition as described in claim 1 that further comprises inorganic particulate thickener.

24. A composition as described in claim 23 wherein the inorganic particulate thickener comprises silica.

25. A composition as described in claim 1 wherein the synthetic ester wax comprises C₁₈-C₃₆ triglyceride wax.

26. A composition as described in claim 1 that is anhydrous.

27. A composition as described in claim 1 wherein the non-volatile oil further comprises an ether oil.

28. A composition as described in claim 1 wherein the non-volatile ester oil comprises an aromatic ester oil.

29. A composition as described in claim 1 wherein the volatile oil comprises silicone oil.

30. A composition as described in claim 1 wherein the volatile oil comprises cyclomethicone.

31. A composition as described in claim 1 wherein the volatile oil comprises cyclopentasiloxane.

32. A composition as described in claim 1 wherein the volatile oil comprises cyclohexasiloxane.

33. A composition as described in claim 1 wherein the amount of natural ester wax, if present, does not exceed 4% by weight, based on the total weight of the composition.

34. A method of reducing or controlling perspiration which comprises applying a composition as described in claim 1 to the underarm area at a dose of from 0.1 to 0.6 grams per underarm.
